(19) United States
(12) Patent Application Publication
(10) Pub. No.: US 2007/0275953 A1
(43) Pub. Date: Nov. 29, 2007

(54) 2-Pyridinone Derivatives, Having Hiv Inhibiting Properties

(75) Inventors: Kiet Le Van, Brussels (BE); Renoit Georges, Louvain-La-Neuve (BE); Laszlo Heyesi, Jambes (BE); Christine Cauvin, Erpent (BE); Sandra Boland, Burdinne (BE); Francois Durant, Namur (BE); Dominique Demonte, Yves-Gomezes (BE); Chris Van Lint, Brussels (BE); Arthu Burny, Gembloux (BE); Alex Bollen, Itterbock (BE); Alain Jacquet, Gorselles (BE); Stephaine De Walque, Foix-Lex-Cavez (BE)

Correspondence Address: MERCHANT & GOULD PC, P.O. BOX 2903, MINNEAPOLIS, MN, 55402-0903, US

(73) Assignees: FACULTES UNIVERSITAIRES NOTRE-DAME DE LA PAIX, Namur (BE); UNIVERSITE LIBRE DE BRUXELLES, Brussels (BE)

(21) Appl. No.: 10/573,143

(22) PCT Filed: Sep. 22, 2004
(86) PCT No.: PCT/BE04/00134

§ 371 Date: May 21, 2007

(30) Foreign Application Priority Data
Sep. 22, 2003 (EP) 03447231.6

(51) Int. Cl. A61K 31/4747; A61P 31/18; C07D 211/86; A61K 31/443; A61K 31/4439; A61K 31/4436; A61K 31/4433; C07D 403/02; C07D 405/02; C07D 409/02

(52) U.S. Cl. 514/220; 514/348; 546/296; 514/278; 514/343; 514/332; 546/15; 546/268.1; 546/261; 546/268.4

(57) ABSTRACT

The present invention relates to 2-Pyridinone derivatives, more specifically 5-ethyl-6-methyl-2-pyridinone derivatives, according to general formula I that inhibit human immunodeficiency virus type 1 (HIV-1) replication and are therefore of interest in the treatment of Acquired Immune Deficiency Syndrome (AIDS). The present invention further relates to the synthesis of said compounds and their use, with or without other pharmaceutical agents, in the treatment of AIDS and viral infections by HIV-1.

### FIELD OF THE INVENTION

The present invention relates to 2-pyridinone derivatives, in particular 5-ethyl-6-methyl-2-pyridinone derivatives, that inhibit HIV, especially human immunodeficiency virus type 1 (HIV-1) replication. They are therefore of interest in the treatment of Acquired Immune Deficiency Syndrome (AIDS). The present invention further relates to the synthesis of said compounds and their use, alone or in combination with other pharmaceutical and/or therapeutic agents, in the treatment of viral infectious diseases like AIDS, especially viral infections by HIV-1.

### BACKGROUND OF THE INVENTION

Human Immunodeficiency Virus (HIV) is the causative agent of AIDS. Two main forms of this virus (HIV-1 and HIV-2) have been identified. HIV—O is a subtype of HIV-1. HIV-1, HIV-2 and HIV—O are all causative agents of AIDS, of which HIV-1 is the most common one. As a retrovirus from the lentivirus family, HIV has its genome in the form of single-stranded RNA.

An essential step of HIV life cycle is therefore the reverse transcription of this single stranded RNA into double-stranded DNA. This process is catalyzed by a virally encoded enzyme known as reverse transcriptase. Numerous reverse transcriptase inhibitors have been used as antiretroviral agents. Most of them can be classified either as nucleoside reverse transcriptase inhibitors (NRTIs), also known as nucleoside analogues, or as non-nucleoside reverse transcriptase inhibitors (NNRTIs) that bind at an allosteric site (referred to as “TIBO site”) some 10 Å from the catalytic site of the reverse transcriptase (RT) as described De Clercq E. et al (New developments in anti-HIV chemotherapy, Biochem. Biophys Acta 2002, 258-275). Most NNRTIs display marked selectivity for HIV-1 inhibition.

### STATE OF THE ART

European patent application EP0462800 describes a first series of pyridinone derivatives and their use in the treatment of HIV-related diseases.

European patent application EP0462808 discloses a series of pyridinone derivatives that are structurally related to those of EP0462800 and also find their use in the treatment of HIV-related diseases.

European patent application EP0481802 describes the preparation of 2-pyridinones and 2-pyridinethiones and their use in the treatment of HIV-related diseases.

International patent application WO97/05113 discloses the preparation of 4-aryl-thio-pyridinones and their use in the treatment of HIV-related diseases.

International patent application WO02/08226 discloses tricyclic 2-pyridinone compounds which are useful as inhibitors of HIV reverse transcriptase.

Published US patent application US2003125340 discloses 3-(Amino- or aminoalkyl)pyridinone derivatives and their use for the treatment of HIV related diseases.

International patent application WO99/55676 discloses the preparation of 3-amino- and 3-aminoalkyl-pyridinone and pyridinethione derivatives and their use in the treatment of HIV-related diseases.

International patent application WO02/24650 and European patent application EP 1 318 995 disclose another series of pyridinone and pyridinethione derivatives displaying HIV inhibiting properties.

The Publication of Dolle et al. (J. Med. Chem. 1995, 38, 4679-4686) discloses a series of 4-aryl-thio-pyridinones.

The present invention provides still further antiviral agents with excellent activity against HIV-1 infections.

### AIMS OF THE INVENTION

The present invention aims to provide new antiviral agents that are able to prevent, inhibit and/or suppress viral infections and that show especially improved inhibitory action towards Human Immunodeficiency Virus type 1 (HIV-1) replication (reversible or irreversible inhibitors active against wild-type and mutant strains).

In particular, the present invention aims to provide such compounds which are non-nucleoside reverse transcriptase inhibitors (NNRTIs), able to block HIV-1 replication, and which do not require metabolic activation (e.g. phosphorylation) to be active.

A preferred aim of the present invention is to obtain such compounds that are irreversible inhibitors, especially compounds that bind irreversibly to the allosteric site of HIV-1 reverse transcriptase (RT).

A further aim of the present invention is to provide such compounds, which can be used in the prevention, suppression and/or the treatment of viral infections, either as pure compounds, as pharmaceutically acceptable salts or as prodrug thereof and/or as ingredient of a pharmaceutical composition, possibly in combination with other antiviral active agents and/or immunomodulators.

A last aim of the present invention is to provide methods of synthesis for such compounds and to provide compounds obtainable by said methods.

### SUMMARY OF THE INVENTION

One aspect of the invention concerns the antiviral compounds of claim 1, preferably antiviral compounds that block the allosteric site of HIV-1 reverse transcriptase, preferably by an irreversible binding (e.g. through a covalent bond). Irreversible antiviral compounds allow a definitive deactivation of this HIV-1 enzyme and therefore a definitive blocking of HIV-1 replication.

Advantageously, these compounds may also be effective in blocking replication of resistant HIV-1 strains that comprise one or more mutations in the (wild type) RT sequence, which may render these strains resistant to existing antiviral compounds.

In particular, the present invention is related to new non-nucleoside reverse transcriptase inhibitor compounds (NNRTIs), which display HIV-1 inhibitory properties, having the general formula I: 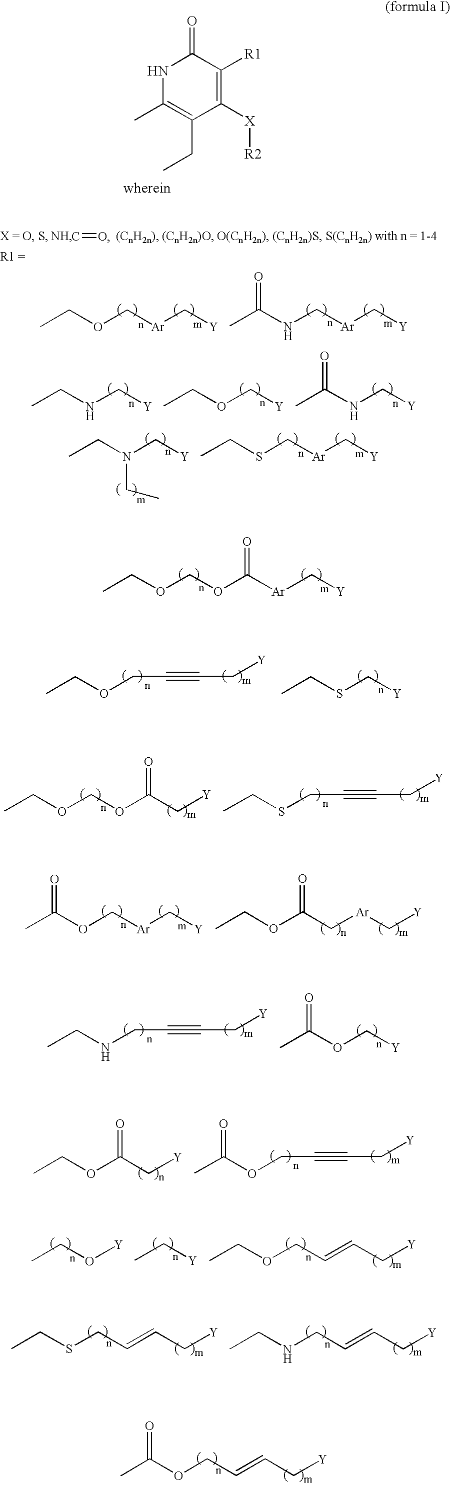
with n, m=0-8
Ar=Aromatic ring selected from: phenyl, pyridyl, thiazolyl, furanyl, thiophenyl, benzofuranyl, benzothiophenyl, benzothiazolyl, imidazolyl, indolyl,
   each optionally substituted with up to 4 substituants selected from:
   halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido
Y═H, halo, alkylamino, dialkylamino, nitrile, hydroxy, C₁₋₆alkyloxycarbonyl, C₁₋₈alkylcarbonyloxy, C₅₋₇ cycloalkyl optionally substituted with up to 4 substituants selected from:
   halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, nitrile;
   or Y can be: 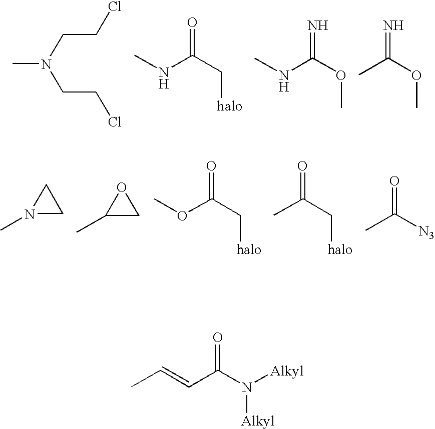
      or Y=alkyl, amino, nitro.
R2=C₇₋₉ cycloalkyl;
   C₅₋₈ cycloalkyl substituted with up to 4 substituants;
   C₅₋₈ cycloalkenyl optionally substituted with up to 4 substituants;
   C₅₋₈ aliphatic heterocycle optionally substituted with up to 4 substituants;
   C₆₋₉ bridged cycloalkyl optionally substituted with up to 4 substituants;
   C₆₋₉ bridged cycloalkenyl optionally substituted with up to 0.4 substituants;
   substituants selected from:
   halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, CN;

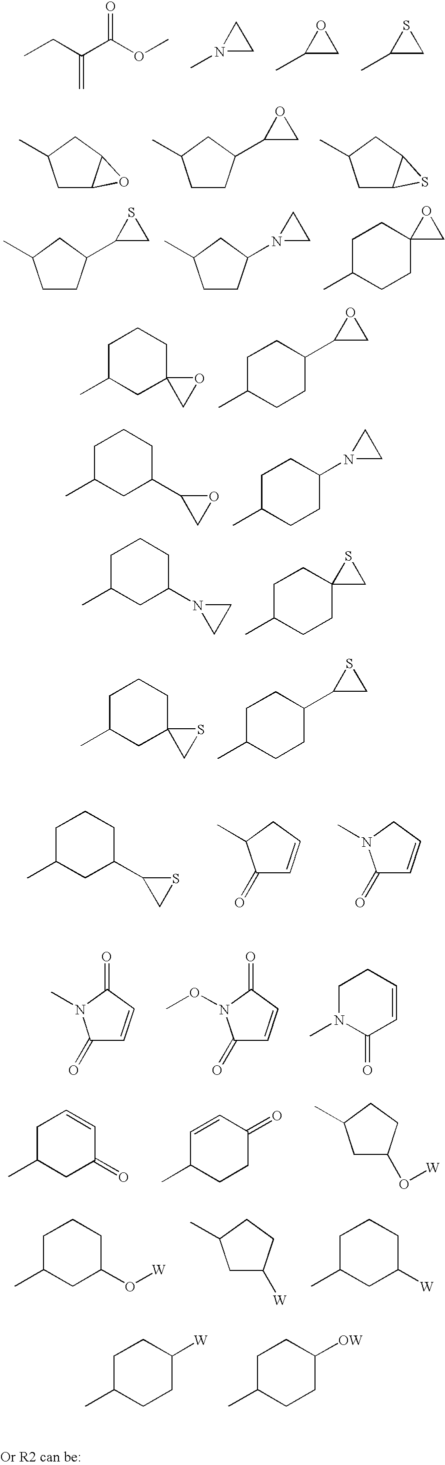

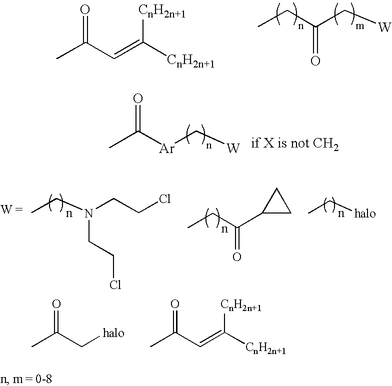

Preferably, in such compounds according to formula 1, X═O or 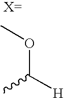
and/or R1 is one selected from the group consisting of CO₂Et, CH₂OH, NO₂, NH₂, CH₂SCOMe, CH₂S(CH₂)₂OH, CH₂S(CH₂)₂OCOCH₂Cl, NMe₂, CH₂N₃, Me, Et, 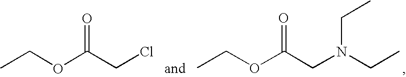
with Me standing for methyl and Et standing for ethyl.

According to an embodiment of the invention, the compound is a compound according to general formula (I) with X and R1 as defined above, and with R2 selected from the group consisting of

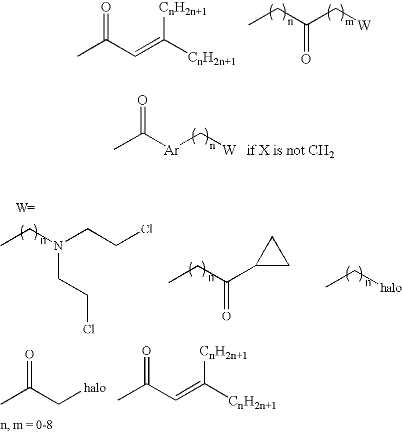

Most preferably, R 2 of said compound is 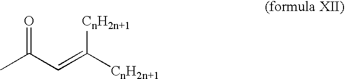
with n=0-8, preferably n=0, 1, 2, 3 or 4, more preferably n=0, 1, or 2 and most preferably n=1.

A most preferred compound is one according to formula I, in which R2 is as given in formula XII, with n preferably=1, and in which X═O and R1 preferably CO₂-Ethyl (CO₂Et).

According to another embodiment, the compound is one according to general formula I in which
R2=C₇₋₉ cycloalkyl;
   C₅₋₈ cycloalkyl substituted with up to 4 substituants;
   C₅₋₈ aliphatic heterocycle optionally substituted with up to 4 substituants;
   C₆₋₉ bridged cycloalkyl optionally substituted with up to 4 substituants;
   substituants selected from
   halo, hydroxy, C₁₋₄ alkyl, C₁₋₄alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, CN;
   or substituents selected from:

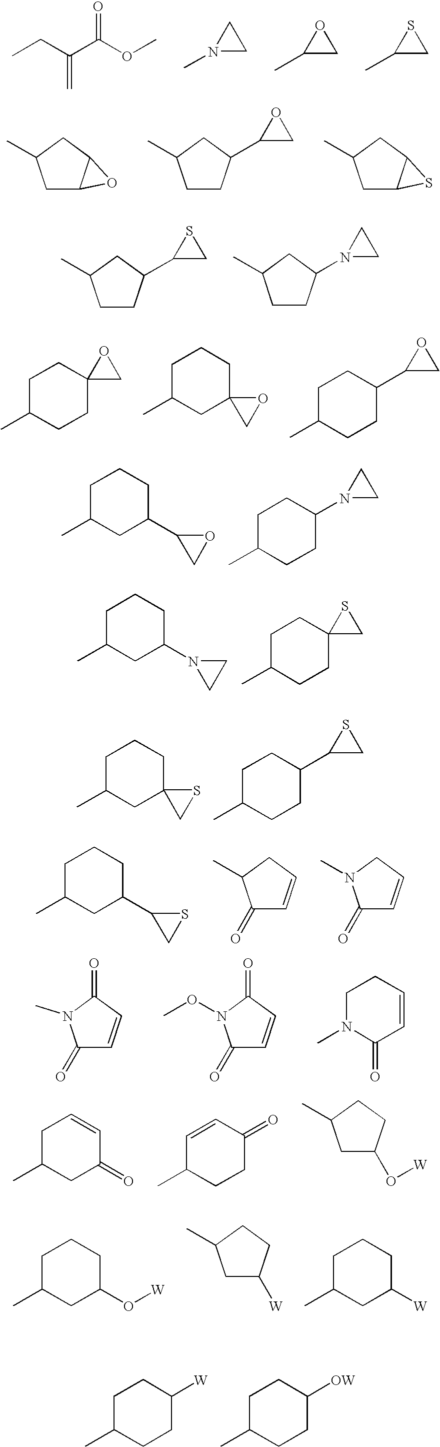

According to another embodiment, the compound is one according to general formula I in which R2 is selected from the group consisting of
C₅₋₈ cycloalkenyl optionally substituted with up to 4 substituants;
C₆₋₉bridged cycloalkenyl optionally substituted with up to 4 substituants;
substituants selected from:
halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, CN;
or substituents selected from:

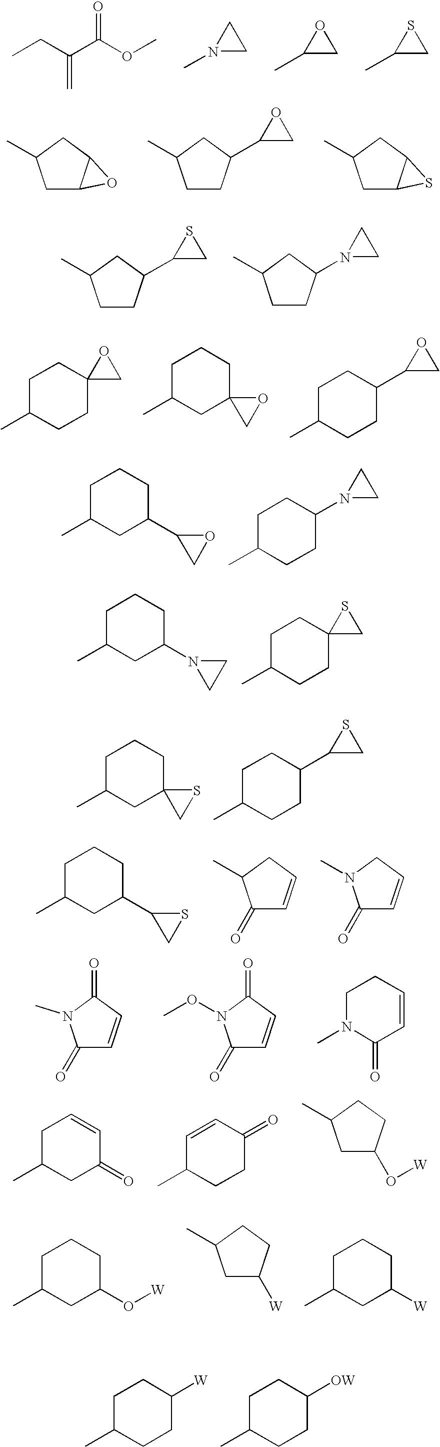

Preferred compounds are those referred to as M18, Z12, Z25, Z30, Z32, Z33, Z37, Z37inv, Z53, Z54, Z55, Z57, Z45inv, Z91inv, Z96inv, Z114, Z121, Z122, Z150, Z153, Z154 and Z167 (see infra, e.g. table 2 wherein X, R1 and R2 are specified for each of said compounds). An aspect of the invention relates to each of these compounds.

The present invention is also related to pharmaceutically acceptable salts or prodrugs of said compounds as well as to a pharmaceutical composition comprising at least one of the different compounds according to the invention (in pure form and/or as acceptable salt and/or as prodrug) and further an adequate pharmaceutical carrier and/or diluent. The compounds according to the invention may be used in combination with any other suitable (known or yet unknown) antiviral compounds, anti-infective agents, immunomodulators, antibiotics and/or vaccines.

Said pharmaceutical composition can find advantageous and efficient use in the prevention, treatment and/or the suppression of viral infections by Human Immunodeficiency Virus type 1 (HIV-1).

Another aspect of the present invention is related to the use of any of the compounds according to the invention (in pure form and/or as salt and/or as prodrug) or the pharmaceutical composition according to the invention as a medicament and/or for the manufacture of a medicament to treat, suppress and/or prevent viral infections induced by Human Immunodeficiency Virus type 1 (HIV-1).

A further aspect of the present invention is related to the preparation method of said compounds as described in detail hereafter.

A last aspect of the present invention concerns a method for obtaining an irreversible anti-HIV-1 compound, which method comprises the steps of:
selecting an anti-HIV-1 compound, preferably a NNRTI, that interacts with a binding site of an HIV-1 enzyme,
introducing a chemical modification in the structure of said anti-HIV-1 compound that allows the formation of at least one covalent bond between the compound and an amino acid of said HIV-1 enzyme.

The obtained (obtainable) irreversible anti-HIV-1 compounds, through the formation of said at least one covalent bond, can bind irreversibly to said HIV-1 enzyme, which preferably is a reverse transcriptase (RT).

Irreversible anti-HIV-1 compound allow a definitive deactivation of the HIV enzyme such as the RT.

Advantageously, an equimolar quantity of said compounds is sufficient for complete deactivation unlike for irreversible compounds, which may be metabolized and/or may be excreted in living cells.

A preferred binding site is the allosteric binding site (TIBO site) of HIV-1 reverse transcriptase.

The chemical modification may imply the introduction of an alkylating function.

Preferably, this chemical modification is the introduction of a chemical function or moiety at position 3 (thus at the level of R1) in a compound according to formula 1.

This may be the introduction of a NH—COCH₂Halo moiety at an existing side group, preferably one at position 3. A preferred halogen is Cl.

Most preferably, the final group (after modification, id est after introduction of the moiety) is one that accords to the formula CH₂X(CH₂)ₙXCO(CH₂)ₘHalo (formula XIII), wherein X is S or O, n is comprised between 1 and 8 and m is comprised between 1 and 8. A preferred halogen is Cl.

A preferred final group is CH₂S(CH₂)₂OCOCH₂Halo. Again, a preferred halogen is Cl.

A most preferred final side group (after modification) is CH₂S(CH₂)₂OCOCH₂Cl.

The above method allows to transform potent (but reversible) anti-HIV-1 compounds, like the NNRTI of the invention, into even more potent compounds by making them irreversible binders (binding compounds) and blockers (blocking agents).

The following examples and specific embodiments are intended for illustration purposes only, and should not be construed as limiting the scope of the invention in any way.

### BRIEF DESCRIPTION OF THE FIGURES

The FIG. 1 represents the synthesis of ethyl 4-[(3,5-dimethylcyclohexyl)oxy]-5-ethyl-6-methyl-pyridine-2(1H)-one-3-carboxylate (compound Z37).

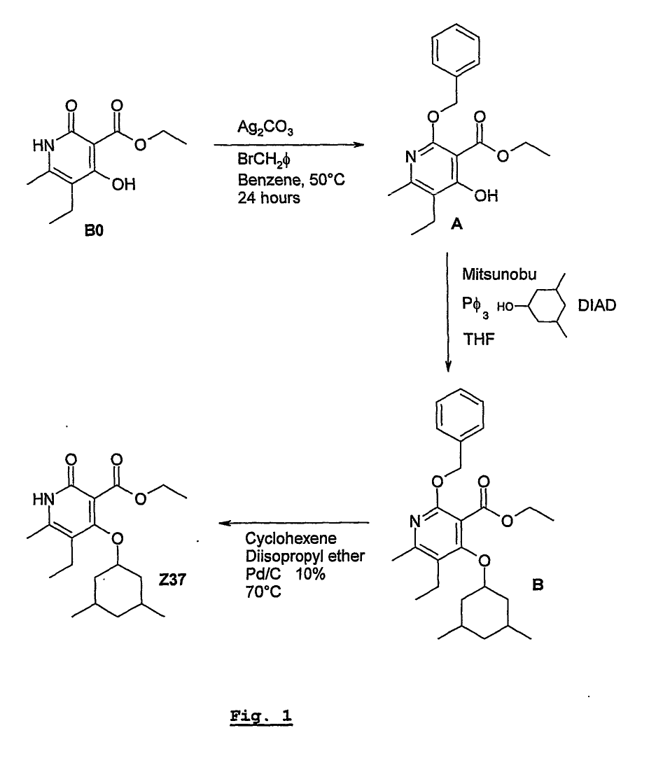

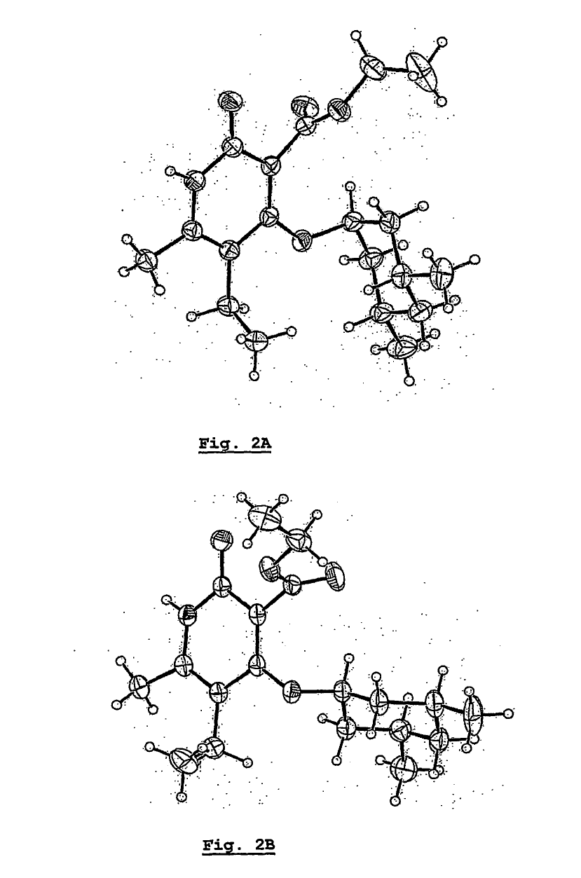

The FIGS. 2A and 2B represent respectively the X-ray structure of compound Z37A and Z37inv.

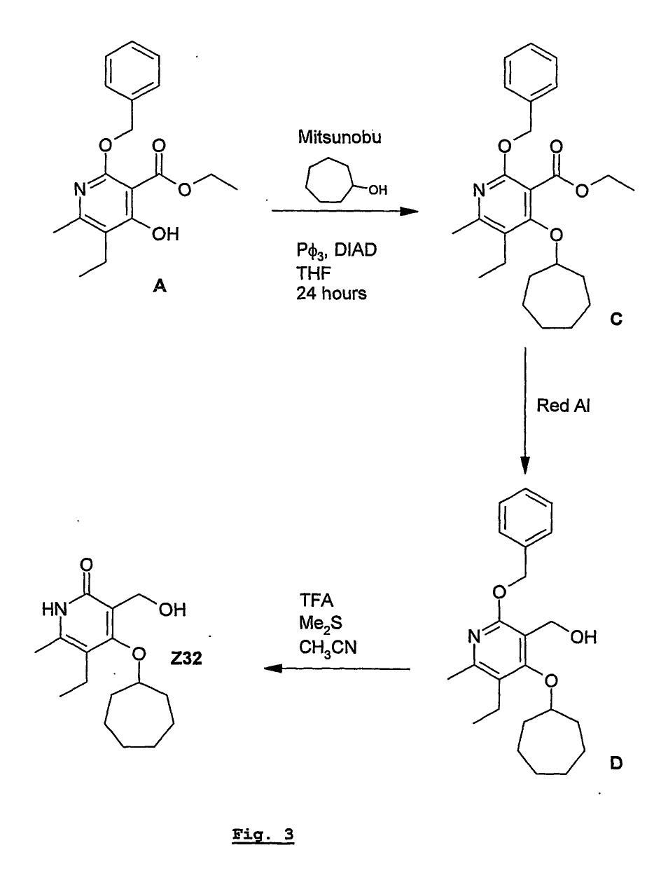

The FIG. 3 represents the synthesis of 4-(cycloheptyloxy)-3-(hydroxymethyl)-5-ethyl-6-methylpyridin-2(1H)-one (compound Z32).

The FIG. 4 represents the synthesis of [4-(cycloheptyloxy)-5-ethyl-6-methyl-2-oxo-1,2-dihydropyridin-3-yl]methyl chloroacetate (compound Z33).

The FIG. 5 represents the synthesis of 2-(dimethylamino)ethyl 4-[(3,5-dimethylcyclohexyl)oxy]-5-ethyl-6-methyl-pyridine-2(1H)-one-3-carboxylate (compound Z53).

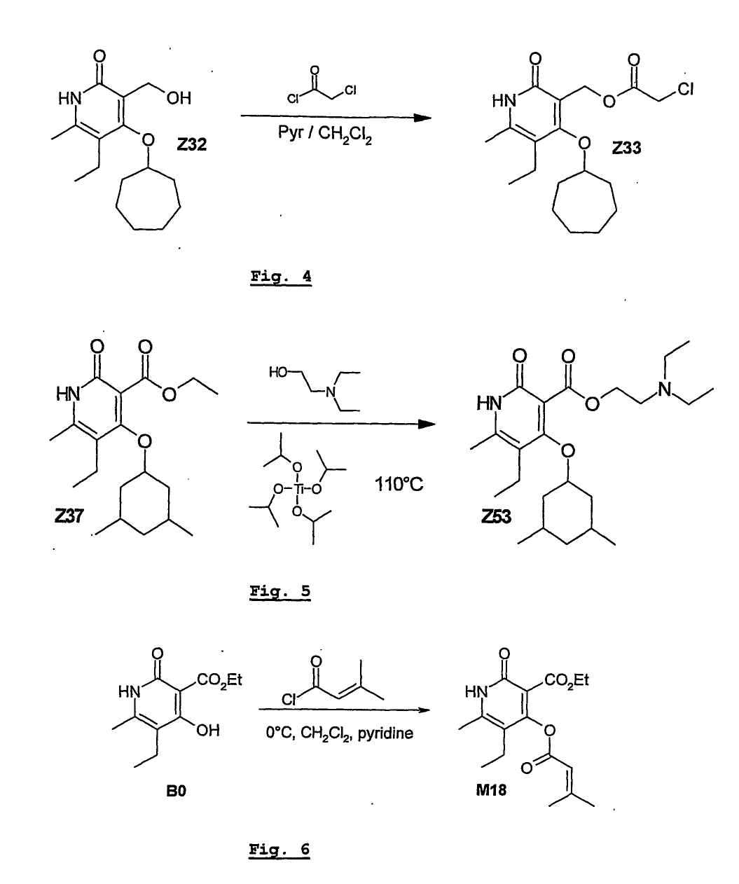

The FIG. 6 represents the synthesis of ethyl 5-ethyl-6-methyl-4-[(3-methylbut-2-enoyl)oxy]-2-oxo-1,2-dihydropyridine-3-carboxylate (compound M18).

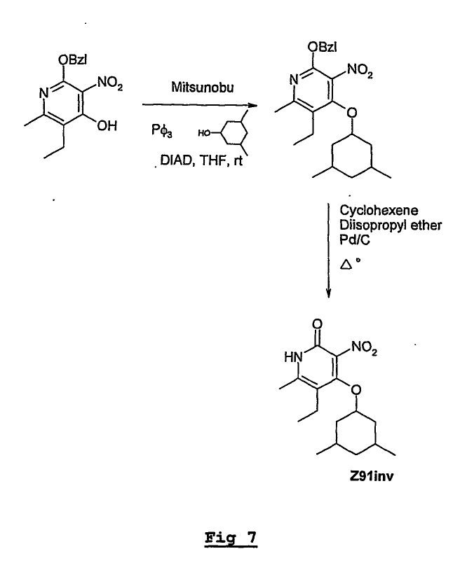

The FIG. 7 represents the synthesis of 3-nitro-5-ethyl-6-methyl-4-[(3,5-dimethylcyclohexyl)oxy]pyridine-2(1H)-one (compound Z91inv).

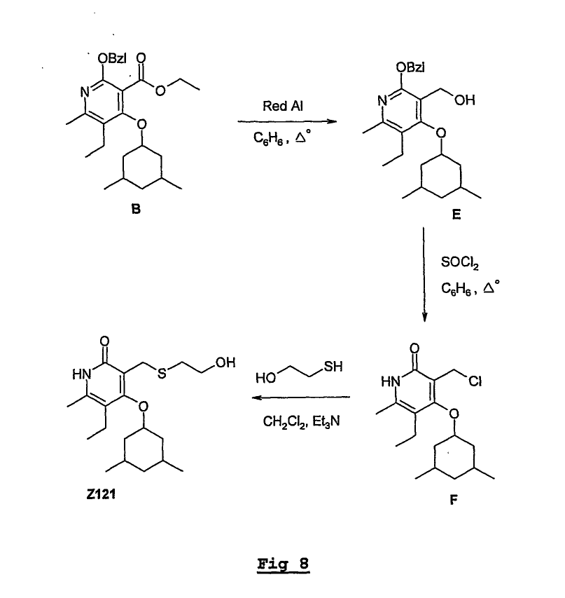

The FIG. 8 represents the synthesis of 5-ethyl-6-methyl-4-[(3,5-dimethylcyclohexyl)oxy]-3-{[(2-hydroxyethyl)sulfanyl]methyl}pyridine-2(1H)-one (compound Z121).

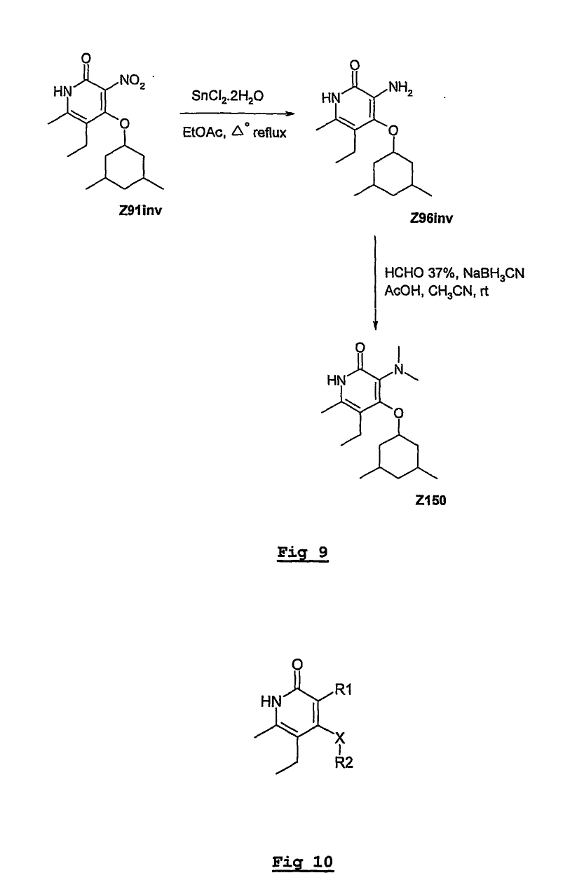

The FIG. 9 represents the synthesis of 3-(dimethylamino)-5-ethyl-6-methyl-4-[(3,5-dimethylcyclohexyl)oxy]pyridine-2(1H)-one (compound Z150).

The FIG. 10 represents general formula I.

### DETAILED DESCRIPTION OF THE INVENTION

Compounds of general formula I (see above) that are described in the present invention behave either as reversible reverse transcriptase inhibitors or as irreversible reverse transcriptase inhibitors. The following two-step mechanism is thought to be involved in irreversible inhibition:
1. reversible binding to the allosteric site (TIBO site) of HIV-1 reverse transcriptase, and
2. formation of a covalent bond with a reactive amino-acid of the TIBO site, leading to irreversible inhibition

Of particular interest are compounds of formula I with a specific substitution in position 4 of the pyridinone ring. Such compounds display an excellent antiviral activity against HIV-1. A particular example hereof is for instance compound Z150, which bears a 3,5-dimethylcyclohexyl moiety as R2 (see general formula above).

Some of the compounds according to the present invention were found to exhibit an excellent antiviral activity against HIV-1 mutant strains that are resistant to one or more antiviral agents active against HIV-1 such as commonly applied NNRTIs like Nevirapine.

### EXAMPLES

### Example 1

### Synthesis of ethyl 4-[(3,5-dimethylcyclohexyl) oxy]-5-ethyl-6-methyl-pyridine-2(1H)-one-3-carboxylate (compound Z37)

Compound Z37 which corresponds to formula II 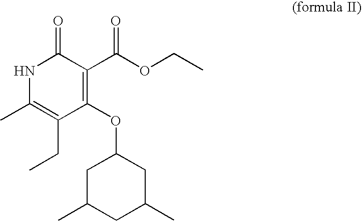
was synthesized, following a three-step protocol, as described below and as illustrated in FIG. 1.
Step 1

ethyl 4-hydroxy 5-ethyl-6-methyl-pyridine-2(1H)-one-3-carboxylate (B0) was synthesized as described by E. Bisagni and al. (% J. Med. Chem. 1995, 38, 4679-4686). Then, benzyl bromide (1.8 g, 10.5 mmol) was added to a stirred suspension of silver carbonate (1.41 g, 5.1 mmol) and B0 (2.25 g, 10 mmol). The mixture was heated (50° C.) overnight then cooled and filtered over celite 521 (Aldrich). The solvent was evaporated and the crude product purified using a silica gel column (e.g. a 60 Å/0.040-0.063 mm ROCC column; eluent: pentane/dichloromethane, 70/30 v/v %), to give intermediate A (2.6 g, 83% yield).

Step 2

In a second step, Diisopropyl azodicarboxylate (DIAD) (0.804 g, 4 mmol) was added drop wise at room temperature to a solution of intermediate A (0.63 g, 2 mmol), triphenylphosphine (PΦ₃) (1.048 g, 4 mmol) and 3,5-dimethylcyclohexanol (0.512 g, 4 mmol) in THF (20 ml). After stirring overnight, the THF was evaporated and the residue was suspended in a mixture of hexane and diethyl ether (50:50 v/v %). The precipitate was filtered off and the organic layer was evaporated. The residue obtained was purified using a silica gel column (e.g. a 60 Å/0.040-0.063 mm ROCC column; eluent: pentane/dichloromethane, 50/50 v/v %), to give intermediate B (0.595 g, 70% yield).

Step 3

In a third step, Pd/C 10% (w/w %) (0.160 g) was added to a solution of intermediate B (0.360 g, 0.89 mmol) in cyclohexane (4 ml) and diisopropyl ether (12 ml). The mixture was heated overnight at 70° C. The precipitate was then filtered off and the organic solvents were evaporated. The product was purified with a silica gel column (e.g. a 60 Å/0.040-0.063 mm ROCC column; eluent: dichloromethane/ethanol, 95/05 v/v %) to give product Z37 as a mixture of stereoisomers (0.238 g, 80% yield, mp (melting point) for the mixture of stereoisomers=108° C.).

The major isomer of the mixture, Z37A (70%) was purified by chiral HPLC (e.g. using a DAICEL chiralpak AD 4.6/250 mm column; eluent: hexane/isopropanol 95/05 v/v %). (mp=122° C.).

A nuclear magnetic resonance (NMR) 1H profile was obtained using an Ex 90 FT NMR spectrometer (Jeol) and gave the following information for compound Z37A:

NMR 1H for Z37A: δ 13 (s, 1H), 4.7 (m, 1H), 4.4 (q, 2H), 2.4 (q, 2H), 2.25 (s, 3H), 2.15-1.6 (m, 8H), 1.35 (t, 3H), 1.0 (t, 3H), 0.85 (d, 6H)

A diastereoisomeric form of Z37A (Z37inv) was obtained by a stereoselective double Mitsunobu reaction (mp=158° C.) (David L. Hugues 1992, “The mitsunobu reaction”, Organic Reaction, 42, 335). Its antiviral activity was found to be higher than the activity observed for either Z37A or the mixture of stereoisomers (see infra).

The stereochemistry of compounds Z37A and Z37inv was checked by X-Ray diffraction using a Enraf-Nonius CAD-4 apparatus (Brucker). The X-Ray diffraction structures of both compounds are given in FIGS. 2 A and B respectively. The crystal data of both compounds, as well as the specific data collection information and applied refinement conditions, are summarized in Table 1.

TABLE 1
Crystal data, data collection and refinement information
for compounds Z37A and Z37inv (symbols used are standard
IUPAC symbols well known in the art)

| | |
|---|---|
| Z37A | Z37inv |

Crystal Data

| | | |
|---|---|---|
| Formula | C₁₉H₂₉NO₄ | C₁₉H₂₉NO₄ |
| MW | 335.43 | 335.43 |
| System, | Triclinic, P-1 | Monoclinic, P21/C |
| space group | | |
| a (Å) | 8.518(1) | 13.488(3) |
| b (Å) | 9.220(2) | 9.064(2) |
| c (Å) | 13.436(1) | 16.584(1) |
| α (°) | 88.906(6) | 90.0 |
| β (°) | 82.053(4) | 110.617(16) |
| γ (°) | 66.369(7) | 90.0 |
| V (Å³) | 956.7(2) | 1897.6(6) |
| Z | 2 | 4 |
| Dₓ (Mg m⁻³) | 1.164 | 1.174 |
| Radiation | Cu K_{α} | Cu K_{α} |
| μ (mm⁻¹) | 0.651 | 0.066 |
| T (K) | 293(2) | 293(2) |
| Crystal | Platelet, colourless | Platelet, colourless |
| Crystal size | 0.34 × 0.30 × 0.13 | 0.45 × 0.34 × 0.25 |

Data collection

| | | |
|---|---|---|
| Diffractometer | Enraf-Nonius CAD-4 | Enraf-Nonius CAD-4 |
| Scan | θ/2θ | θ/2θ |
| Absorption | Analytical | None |
| correction | Tₘᵢₙ = 0.809, Tₘₐₓ = 0.920 | |
| Measured | 4012 | 4075 |
| reflections | | |
| Independent | 3764 | 3907 |
| reflections | | |
| Reflections with | 3067 | 3223 |
| I > 2 σ(I) | | |
| Rᵢₙₜ | 0.0137 | 0.0967 |
| θₘₐₓ | 71.97 | 74.98 |
| h | −9 −> 10 | 0 −> 16 |
| k | 0 −> 11 | 0 −> 11 |
| l | −16 −> 16 | −20 −> 19 |

Refinement

| | | |
|---|---|---|
| Refinement on | F² | F² |
| R[F² > 2 σ(F²)] | 0.0526 | 0.0825 |
| wR (F²) | 0.1759 | 0.2150 |
| S | 1.462 | 1.689 |
| Number of | 3764 | 3907 |
| reflections | | |
| Number of | 223 | 221 |
| parameters | | |
| (Δ/σ)ₘₐₓ | 0.013 | 0.001 |
| Δρₘₐₓ | 0.289 | 0.421 |
| Δρₘᵢₙ | −0.299 | −0.335 |

### Example 2

### Synthesis of 4-(cycloheptyloxy)-3-(hydroxymethyl)-5-ethyl-6-methylpyridin-2(1H)-one (compound Z32)

Compound Z32 which corresponds to formula III 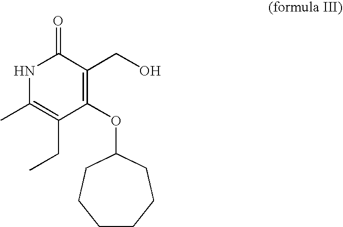
was synthesized, following a three-step protocol, as described below and as illustrated in FIG. 3.
Step 1

In a first step, Diisopropyl azodicarboxylate (DIAD) (0.804 g, 4 mmol) was added drop wise at room temperature to a solution of above-described intermediate A (0.63 g, 2 mmol), triphenylphosphine (PΦ3) (1.052 g, 4 mmol) and cycloheptanol (0.456 g, 4 mmol) in THF (20 ml). After stirring overnight, the THF was evaporated and the residue was suspended in a mixture of hexane and diethyl ether (50:50 v/v %). The precipitate was filtered off and the organic layer was evaporated. The residue obtained was purified using a silica gel column (e.g. a 60 Å/0.040-0.063 mm ROCC column; eluent:pentane/dichloromethane, 50/50 v/v %), to give intermediate C (0.575 g, 70% yield).

Step 2

In a second step, Red-Al (2.3 ml, 7.6 mmol) was suspended in benzene (10 ml). Intermediate C (1.77 g, 4.3 mmol) was added to this solution, at 0° C. The mixture was heated at 75° C. for 2 hours and then cooled again at 0° C. After addition of a solution of 20% sulphuric acid, the aqueous layer was extracted with dichloromethane. The organic extracts were collected and washed with brine.

The crude product was chromatographed on silica gel column (e.g. a 60 Å/0.040-0.063 mm ROCC column; eluent: dichloromethane/pentane, 50/50 v/v %) to afford intermediate D (1.507 g, 95% yield).

Step 3

In a third step, Intermediate D (1.5 g, 4 mmol) was then dissolved in a mixture of acetonitrile (10 ml) and dimethyl sulfide (2 ml). Trifluoroacetic acid (1 ml) was then added to this mixture, at 0° C. After stirring at room temperature for 3 hours, the solvents were evaporated. The residue was dissolved in dichloromethane and washed with a solution of saturated NaHCO₃.

After drying with MgSO₄ and evaporation of the solvent, recrystallisation from ethyl acetate/hexane gave pure product Z32 as white crystals (mp=146° C.)

A nuclear magnetic resonance (NMR) 1H profile was obtained using an Ex 90 FT NMR spectrometer (Jeol) and gave the following information for compound Z32:

NMR 1H for Z32: δ 13 (s, 1H), 4.6 (s, 2H), 4.1 (m, 1H), 2.44 (q, 2H), 2.31 (s, 3H), 1.7 (m, 12H), 1.1 (t, 3H)

### Example 3

### Synthesis of [4-(cycloheptyloxy)-5-ethyl-6-methyl-2-oxo-1,2-dihydropyridin-3-yl]methyl chloroacetate (compound Z33)

Compound Z33 which corresponds to formula IV 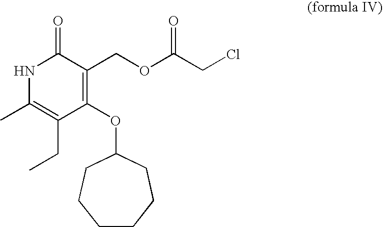
was synthesized from compound Z32 as described below and as illustrated in FIG. 4. Compound Z32 (165 mg, 0.6 mmol) was dissolved in dichloromethane (2 ml) and pyridine (50 μl). Chloroacetyl chloride (50 μl) was added to this mixture cooled at 0° C. After stirring for 3 hours at 0° C., HCl (1 ml, 1N) and dichloromethane (10 ml) were added to this solution.

The organic layer was washed with a saturated NaCl solution and the crude product purified using a silica gel column (eluent: MeOH/CH₂Cl₂, 1/9) to give the crystalline product Z33 (0.160 g, Yield: 75%, mp=123° C.).

A nuclear magnetic resonance (NMR) 1H profile was obtained using an Ex 90 FT NMR spectrometer (Jeol) and gave the following information for compound Z33:

NMR 1H for Z33: δ0.13 (s, 1H), 5.2 (s, 2H), 4.2 (m, 1H), 4.0 (s, 2H), 2.5 (m, 2H), 2.3 (s, 3H), 1.7 (m, 12H), 1.1 (m, 3H)

### Example 4

### Synthesis of 2-(dimethylamino)ethyl 4-[(3,5-dimethylcyclohexyl)oxy]-5-ethyl-6-methyl-pyridine-2(1H)-one-3-carboxylate (compound Z53)

Compound Z53 which corresponds to formula V 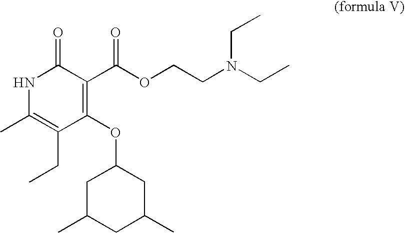
was synthesized from compound Z37 as described below and as illustrated in FIG. 5. To a solution of Z37 (0.167 g, 0.5 mmol) in N,N-diethylethanolamine (3 ml) was added a catalytic amount of tetraisopropyl titanate (c.a. 30 mg). The mixture was stirred overnight at 110° C.

The solvent was then evaporated under vacuum and the residue was extracted with dichloromethane. The crude product was purified with a silica gel column (eluent: dichloromethane/ethanol, 90/10) to give product Z53 (0.121 g, 60% yield, oil).

A nuclear magnetic resonance (NMR) 1H profile was obtained using an Ex 90 FT NMR spectrometer (Jeol) and gave the following information for compound Z53:

NMR 1H for Z53: δ 12.8 (s, 1H), 4.7 (m, 1H), 4.3 (t, 2H), 2.9-2.4 (m, 6H), 2.3 (s, 3H), 2.1-1.4 (m, 8H), 1.25-0.7 (m, 17H).

### Example 5

### Synthesis of ethyl 5-ethyl-6-methyl-4-[(3-methylbut-2-enoyl) oxy]-2-oxo-1,2-dihydropyridine-3-carboxylate (compound M18)

Compound M18 which corresponds to formula VI 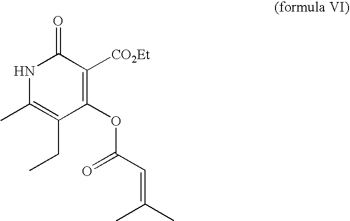
was synthesized as described below and as illustrated in FIG. 6. Intermediate B0 (0.338 g, 1.5 mmol) was dissolved in dichloromethane (10 ml) and pyridine (1 ml). 3,3-dimethyl acryloyl chloride (0.360 g, 3.0 mmol) was added to this solution at 0° C. and the solution was stirred overnight. The solvents were then removed in vacuo. Purification by silica gel chromatography (e.g. a 60 Å/0.040-0.063 mm ROCC column; eluent: ethanol/dichloromethane, 98/02 v/v %) gave product M18 (0.270 g, 60% yield, mp=166° C.).

A nuclear magnetic resonance (NMR) 1H profile was obtained using an Ex 90 FT NMR spectrometer (Jeol) and gave the following information for compound M18:

NMR 1H for M18: δ 13 (s, 1H), 5.9 (s, 1H), 4.2 (q, 2H), 2.5 (m, 2H), 2.3 (s, 3H), 2.2 (s, 3H), 2.0 (s, 3H), 1.3 (t, 3H), 1.1 (t, 3H)

Compounds Z12, Z25, Z30, Z54 and Z55 were synthesized in a similar way as compound Z37. The protocol is similar except for the alcohol used in the second step, which is 2-chlorocyclohexanol for Z12, cycloheptanol for Z25, 3-methylcyclohexanol for Z30, cyclooctanol for Z54 and 4-ethylcyclohexanol for Z55.

### Example 6

### Synthesis of 3-nitro-5-ethyl-6-methyl-4-[(3,5-dimethylcyclohexyl)oxy]pyridine-2(1H)-one (compound Z91inv)

Compound Z91inv, which corresponds to formula I below, was synthesized in a similar way as compound Z37 as illustrated in FIG. 7.

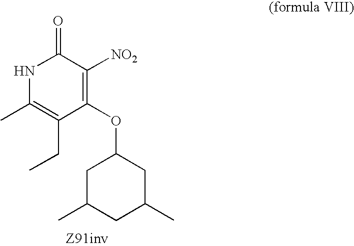

### Example 7

### Synthesis of 5-ethyl-6-methyl-4-[(3,5-dimethylcyclohexyl)oxy]-3-([(2-hydroxyethyl)sulfanyl]methylpyridine-2(1H)-one (compound Z121)

Compound Z121, which corresponds to formula IX below, was synthesized following a three-step protocol, as described below and as illustrated in FIG. 8. 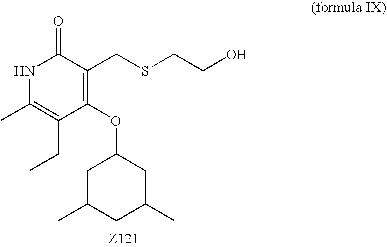
Step 1

Intermediate E was synthesized from B in a similar way as compound Z32 (step 2).

Step 2

Thionyl chloride (0.5 ml, 6.8 mmol) was added to a solution of intermediate E (0.383 g, 1 mmol) in benzene (15 ml). The mixture was heated at reflux for 4 hours. After evaporation of the solvent, the residue was precipitated in 30 ml of hexane giving intermediate F (0.155 g, 50% yield). Intermediate F was unstable and must be used immediately for the next step.

Step 3

The mixture of intermediate F (0.155 g, 0.5 mmol), 2-mercaptoethanol (0.156 g, 2 mmol) and triethylamine (0.2 ml) in dichloromethane (10 ml) was stirred at room temperature for 24 hours. After evaporation of the solvent, the residue was extracted by dichloromethane and neutralised by hydrochloric acid (0.1M). The residue was purified on silica gel column (e.g. a 60 Å/0.040-0.063 mm ROCC column; eluent: dichloromethane/ethanol, 95/5 v/v %) to give compound Z121 (0.088 g, 50% yield).

NMR 1H for Z121: δ0.13 (s, 1H), 5.2 (s, 1H), 4.2 (m, 1H), 4 (m, 4H), 2.95 (m, 2H), 2.4 (q, 2H), 2.25 (s, 3H), 2-1 (m, 11H), 0.9 (d, 6H)

### Example 8

### Synthesis of 3-(dimethylamino)-5-ethyl-6-methyl-4-[(3,5-dimethylcyclohexyl)oxy]pyridine-2(1H)-one (compound Z150)

Compound Z150, which corresponds to formula X below, was synthesized, following a two-step protocol, as described below and as illustrated in FIG. 9. 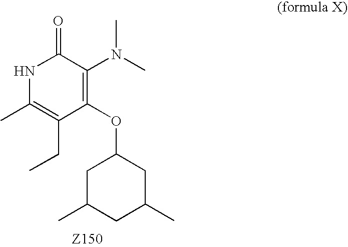
Step 1

A mixture of Z91inv (0.550 g, 1.786 mmol) and tin(II) chloride dihydrate (2 g, 8.88 mmol) in ethylacetate (30 ml) was heated under reflux for 3 hours. After cooling at 0° C. and adding ice water, the suspension was basified with a solution of 10% sodium carbonate. The filtrate was evaporated and the residue was purified on silica gel column (e.g. a 60 Å/0.040-0.063 mm ROCC column; eluent: dichloromethane/ethanol, 95/5 v/v %) giving compound Z96inv (0.397 g, 80% yield).

NMR 1H for Z96inv: δ 12.6 (s, 1H), 4.2 (m, 1H), 3.9 (m, 2H), 2.4 (q, 2H), 2.25 (s, 3H), 2-1 (m, 11H), 0.9 (d, 6H)

Step 2

A mixture of Z96inv (0.150 g, 0.54 mmol), aqueous formaldehyde 37% (0.8 ml, 10 mmol), sodium cyanoborohydride (0.150 g, 2.4 mmol), acetic acid (0.2 ml, 3.4 mmol) and acetonitrile (10 ml) was stirred at room temperature for 24 hours. After evaporation of the solvent, the residue was extracted with dichloromethane (3×30 ml) and neutralised by aqueous sodium hydroxide 10%. The residue was purified on silica gel column (e.g. a 60 Å/0.040-0.063 mm ROCC column; eluent: dichloromethane/ethanol, 95/5 v/v %) giving compound Z150 (0.077 g, 50% yield).

NMR 1H for Z150: δ 12.2 (s, 1H), 4.8 (m, 1H), 2.8 (s, 6H), 2.4 (q, 2H), 2.25 (s, 3H), 2-1 (m, 11H), 0.9 (d, 6H)

Compound Z45inv was synthesized from intermediate E in a similar way as compound Z32 (third step).

Compound Z122 was synthesized from Z121 in a similar way as compound Z33.

Table 2 provides information on the structure and physico-chemical properties of specific compounds according to the invention, such as the nature of the side groups R1 and R2 and of the spacer X, the melting point of the compound and its molecular weight.

TABLE 2
Structure and physico-chemical properties of
specific compounds (N°) according to the invention

| | | | | | Molecular |
|---|---|---|---|---|---|
| N° | X | R1 | R2 | mp (° C.) | Weight |
| M18 | O | CO₂Et | 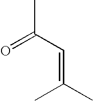 | 166 | Calculated: 307 Measured: 307 |
| Z12 | O | CO₂Et | 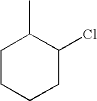 | oil | Calculated: 341 Measured: 341 |
| Z25 | O | CO₂Et | 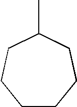 | 112 | Calculated: 321 Measured: 321 |
| Z30 | O | CO₂Et | 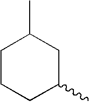 | oil | Calculated: 321 Measured: 321 |
| Z32 | O | CH₂OH | 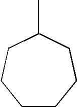 | 146 | Calculated: 279 Measured: 279 |
| Z33 | O | 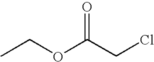 | 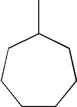 | 123 | Calculated: 355 Measured: 355 |
| Z37 | O | CO₂Et | 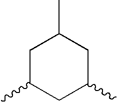 | 108 | Calculated: 335 Measured: 335 |
| Z53 | O | 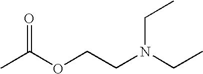 | 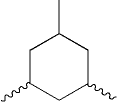 | oil | Calculated: 406 Measured: 406 |
| Z54 | O | CO₂Et | 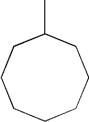 | 104 | Calculated: 335 Measured: 335 |
| Z55 | O | CO₂Et | 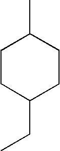 | 100 | Calculated: 335 Measured: 335 |
| Z57 | 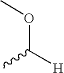 | CO₂Et | 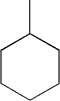 | oil | Calculated: 335 Measured: 335 |
| Z45inv | O | CH₂OH | 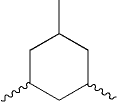 | 157-158 | Calculated: 293 Measured: 293 |
| Z91inv | O | NO₂ | 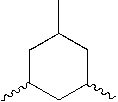 | 206-207 | Calculated: 308 Measured: 308 |
| Z96inv | O | NH₂ | 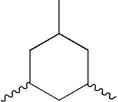 | 251-252 | Calculated: 278 Measured: 278 |
| Z114 | O | CH₂SCOMe | 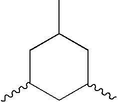 | 140-143 | Calculated: 351 Measured: 351 |
| Z121 | O | CH₂S(CH₂)₂OH | 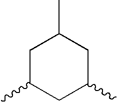 | 133-134 | Calculated: 353 Measured: 353 |
| Z122 | O | CH₂S(CH₂)₂OCOCH₂Cl | 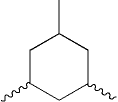 | Oil | Calculated: 429 Measured: 429 |
| Z150 | O | NMe₂ | 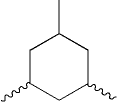 | 129-130 | Calculated: 306 Measured: 306 |
| Z153 | O | CH₂N₃ | 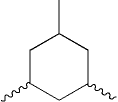 | 138-140 | Calculated: 318 Measured: 318 |
| Z154 | O | Me | 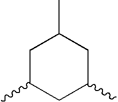 | 149-150 | Calculated: 277 Measured: 277 |
| Z167 | O | Et | 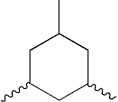 | 160-161 | Calculated: 291 Measured: 291 |

Oil = viscous liquid formation

The following examples demonstrate that the compounds of the present invention are very efficient NNRTIs with HIV-1 inhibiting activity.

This is illustrated via in vitro reverse transcriptase assays and via anti-HIV assays using P4, TMZ-b1 and MT4 cell lines and PBMC. Both the P4 and TMZ-b1 cell lines contain in their genomes the bacterial LacZ gene under the transcriptional control of HIV-1 LTR elements. In these cells, the expression level of the β-galactosidase gene is proportional to the viral replication. The P4 cells express at their surface the CD4 protein, used as a receptor by HIV. The TMZ-b1 cells express at their surface both the CD4 and CCR5 proteins. CCR5 is used as a co-receptor by HIV-1. The presence of both the receptor and co-receptor at their cell surface make the TMZ-b1 cells much more sensitive to infection by the virus compared to the P4 cells. The MT-4 cell line is also widely used to assess the efficacy of drugs against HIV. The MT-4 cell line is derived from CD4⁺ T-lymphocytes chronically infected with Human T-cell Lymphotrophic Virus-1 (HTLV-1). These cells rapidly die upon infection by HIV. In this system cellular viability is inversely proportional to viral replication. Peripheral Blood Mononuclear Cells (PBMCs) isolated from the blood of non infected donor contain primary CD4⁺ T-lymphocytes. These cells are one of the main targets of HIV in infected individuals. In this system viral replication is measured by quantifying the viral capsid protein p24 in supernatants of infected cells cultures.

For the in vitro inhibition studies of the HIV-1 reverse transcriptase activity, stock solutions of the compounds of the present invention were prepared in dimethyl sulfoxide at a final concentration of 10 mM and kept at room temperature. Nevirapine was purchased from Boehringer Ingelheim. Efavirenz was received from the NIH AIDS Research and Reference Reagent Program.

For the antiviral and the cytotoxicity assays, the drugs were diluted in complete DMEM medium. In these experiments, drugs were diluted in triplicate wells in a 96-well plate in six, 5 fold serial dilutions.

### Example 9

### Effect of the Compounds According to the Invention on the HIV-1 In Vitro Reverse Transcriptase Activity

HIV-1 Reverse Transcriptase Activity:

In vitro inhibition studies used a fixed-time assay for HIV-1 reverse transcriptase RNA dependent DNA polymerase activity. RT was purchased from Calbiochem (ref CAL382129-500). One unit of RT corresponds to the amount of enzyme which incorporates one nanomole of [³H]TTP in 10 minutes at 37° C.

Assays were performed in a final volume of 50 μl. The mixture contained 0.125 units of RT, 10 mM MgCl₂, 2 mM DTT, 50 mM Tris pH 8.3, SOMM KCl, 1 μg/μl BSA, 0.01% triton X100, 20 μg/ml (0.4 A260/ml) poly(rC)-oligo(dG)₁₂₋₁₈, 1 μCi [³H]dGTP and 1 μl of the inhibitor (dissolved in dimethyl sulfoxide, DMSO). Reaction mixtures were incubated at 37° C. for 10 min. The incorporation rate was determined by a standard trichloroacetic acid precipitation procedure (adapted from Current protocols in molecular biology. Eds Wiley, MGH Harvard medical school) and liquid scintillation counting using a Wallac scintillation counter.

Results for some of the compounds according to the invention are summarized in Table 3. The in vitro activity of the compounds, at a final concentration of 10 μM, on the reverse transcriptase (RT) activity of HIV-1 is derivable from the relative (%) reduction in RT activity. Hereby, the RT activity in the absence of any of the compounds is set at 100%.

From table 3 it is evident that all the compound tested were able to reduce the in vitro RT activity by at least about 30%. Most of the compounds tested were able to reduce the in vitro activity by at least 50 to 60%. The most active compounds (Z91inv, Z114, Z150, Z153 . . . ) reduced the activity by 99-100%. This reduction of RT activity is better than the one observed with nevirapine and comparable to the one observed with efavirenz, both common NNRTI.

TABLE 3
In vitro residual RT activity after addition of some compounds
(N°) belonging to the invention. Comparison with Nevirapine
and Efavirenz, two common RT inhibitors

| | Relative (%) RT activity in vitro |
|---|---|
| N^{o} | (compound added at 10 μM) |
| M18 | 70.8 |
| Z12 | 63.7 |
| Z25 | 34.6 |
| Z30 | 22.0 |
| Z32 | 44.0 |
| Z33 | 42.0 |
| Z37 | 8.3 |
| Z37A | 7.6 |
| Z37B | 5.6 |
| Z37inv | 5.5 |
| Z45inv | 12.1 |
| Z53 | 34.3 |
| Z54 | 24 |
| Z55 | 40.3 |
| Z57 | ND |
| Z91inv | 0 |
| Z96inv | 25.4 |
| Z114 | 0 |
| Z121 | 1.4 |
| Z122 | 39.1 |
| Z150 | 0.9 |
| Z153 | 0.7 |
| Z154 | 1.6 |
| Nevirapine | 16.7 |
| Efavirenz | 1.7 |

In vitro tests also show that inhibition of RT by the compound Z122 increases with respect to the time of incubation. Z122 seems to be an irreversible inhibitor of the reverse transcriptase. In agreement with this hypothesis, we observed in the same experiments that the activity of the alcohol derivative Z121, which lacks the alkylating function, is independent of the preincubation time with the RT.

After the preincubation, the unbound Z122 and the RT were separated using a micro-spin desalting column. Following this treatment, we did not observe any increase in the RT activity. However, the same experiment performed with the Z121 molecule resulted in a significant increase in the RT activity following the separation step. These data are consistent with the formation of a covalent link between the Z122 compound and the reverse transcriptase.

### Example 10

### Anti-HIV-1 Activity (EC50 Value Expressed in μM) on P4, TZM-b1, MT4 Cell Lines and PBMC and Cytotoxicity (CC50 Value Expressed in μM) of Some of the Compounds According to the Invention

Production of Viral Stocks:

Wurzburg Jurkat T cells (subclone JR) were transfected with 10 μg of the circularly permuted infectious molecular clone HIV_{NL4-3} (Adachi et al., 1986. J. Virol, 59 (2), p 284-291). Two days later, co-cultivation with SupT1 cells (a human T-cell lymphoma cell line) was initiated to facilitate rapid production of progeny virions. Production of virus was measured by using the Innotest HIV Antigen mAb p24 kit (Innogenetics). At the peak of production cultures were harvested and filtered. The virus stocks were stored at −80° C. until used.

In order to introduce mutation(s) at the level of the pol gene coding for the RT in the pNL4-3 plasmid containing the complete viral genome (Adachi et al., 1986. J. Virol., Vol. 59 p 284-291) we used the “Quick change mutagenesis kit” (Stratagene). The following mutations were separately introduced in pNL4-3: L100I, K103N, V108I, Y181C, Y188C, and the double mutations K103N/V108I. These mutations were chosen because of the resistance that they confer to existing NNRTI. The production procedure of the mutant viral stocks is identical to the one described above.

P4 Cell Line:

Anti-HIV activity and cytotoxicity of the compounds were tested on a P4 cell line. The P4 cell line (Clavel & Charneau, 1994. J. Virol., Vol. 68 p 1179-1185) was provided by Dr. François Clavel (Unité de recherche antivirale de l'hôpital Xavier Bichat Paris: Inserm). The P4 cells were cultured in complete DMEM medium supplemented with 10% fetal bovine serum (FBS), 0.5% of Penicillin/Streptomycin and G418 at 0.5 mg/ml. Exponentially growing cells were trypsinized, centrifuged and split twice weekly at 5.10⁴ cells/ml.

TZM-b1 Cell Line:

Anti-HIV activity and cytotoxicity of the compounds were tested on the TZM-b1 cell line. The TZM-b1 cell line (Wei et al. 2002. Antimicrob. Agents Chemother. Vol. 46, p 1896-1905) was received from the NIH AIDS Research and Reference Reagent Program. These cells were cultured in complete DMEM medium supplemented with 10% fetal bovine serum (FBS), and 1% Penicillin/Streptomycin. Exponentially growing cells were trypsinized, centrifuged and split twice weekly at 5 10⁴ cells/ml.

MT-4 Cell Line:

Anti-HIV activity of the compounds was tested on MT-4 cell line. The MT-4 cell line (Larder et al. 1989. Sciences, Vol. 243 p 1731) was received from the NIH AIDS Research and Reference Reagent Program. These cells were cultured in RPMI medium supplemented with 10% fetal bovine serum (FBS), and 1% Penicillin/Streptomycin. The cultures were split regularly to keep cells densities between 0.3 to 1.2 10⁶ cells/ml.

Peripheral Blood Mononuclear Cells:

Anti-HIV activity of the compounds was tested on Peripheral Blood Mononuclear Cells (PBMCs). These cells were separated from the blood of healthy donor using established procedures. PBMCs were cultured in RPMI medium supplemented with 10% fetal bovine serum (FBS), 1% Penicillin/Streptomycin and 20 U/ml of Interleukin-2.

Cytotoxicity of the Compounds

The 50% cytotoxic concentration (CCso) was determined using a protocol adapted from Pauwels et al. (1988. J. Virol. Methods 20(4):309-21). Briefly, flat bottom 96-well plates were filled with 50 μl of complete medium containing 5.10³ P4 cells. 2 hours later 50 μl of drug solution were added to the cells. Drugs (dissolved in DMEM, see above) were diluted in six, 5-fold serial dilutions from stock solutions in triplicate wells of a 96-well plate. Cells and compounds were incubated at 37° C. in growth medium for 3 days. Cell viability was determined by MTT assays using the Roche Cell Proliferation KIT. The absorbance (λ=570 nm) was measured on a Benchmark™ Microplate Reader (Biorad) and compared with 12 cell control replicates (no drug added). Each assay was performed at least three times for a total of at least nine replicate wells. This method detects both cytostatic and cytolytic effects of drugs.

Anti-HIV Assay

The P4 cells are HIV-infectible Hela-CD4 cells that carry the bacterial lacZ gene under the control of the HIV-1 long terminal repeat (LTR). In this cell line, transcription of the LacZ gene is driven by the HIV-1 LTR. As such, the cytoplasmic accumulation of β-galactosidase is strictly dependent on the presence of the HIV transactivator Tat produced during the intracellular viral replication (Clavel & Charneau, 1994. J. Virol., Vol. 68, p. 1179-1185). In other words, in this system, the expression level of the β-galactosidase gene is proportional to the viral replication.

Briefly, in the anti-HIV assay 100 Ål of P4 cells were plated in 96-well plate at a concentration of 0.4 10⁵ cells/ml and incubated at 37° C., 5% CO₂. After 48 h, the medium was removed and 100 Ål of the different drugs dilutions were added to the cells. Four hours after the addition of the drugs all cells were infected with equal amount of cell-free virus, corresponding to 100 ng of HIV p24 antigen. After 48 h of incubation at 37° C., 5% CO₂, β-galactosidase activity was measured using chlorophenol red-β-D-galactopyranoside assays.

The absorbance was measured on a Benchmark™ Microplate Reader (Biorad) (λ=570 nm) and compared with 12 cell control replicates (no virus or drug added) and 12 virus control wells (no drug added). Each assay was performed a minimum of three times. The 50% effective concentration (Ec₅₀) was calculated from each dose response curve using the CurveExpert 1.3 software. As Nevirapine activity corresponds to its published values (10-100 nM), the data are consistent with other measures of viral replication.

The anti-HIV assay with TZM-b1 cells is essentially the same as described above with P4 cells. The main difference between the two cell lines is that the TZM-b1 cells express at their surface both the CD4 and CCR5 proteins, acting as receptor and co-receptor for HIV entry, respectively (Wei et al. 2002. Antimicrob. Agents Chemother. Vol. 46, p 1896-1905). This feature makes the cells very sensitive to infection by the virus. The TMZ-b1 cells were infected with equal amount of cell-free virus, corresponding to 10 ng of HIV p24 antigen. This amount is ten times lower than the amount used to infect the P4 cells.

The MT-4 cells rapidly die upon infection by HIV. In this system there is an inverse correlation between cells survival and the amount of viral replication.

Briefly, in the anti-HIV assay exponentially growing MT-4 cells were infected with HIV-1. 100 μl of infected MT-4 cells were seeded in 96-well plate at a concentration of 0.4 10⁵ cells/ml, and 100 μl of the different drugs dilutions were added to the cells. After 4 days of incubation at 37° C., 5% CO₂, cells survival was measured using MTS (3-(4-5-dimethylthiazol-2-yl)-5-(−(3-carboxymethoxyphenyl)-2-(4-sulfophenyl)-2H-tetrazolium) assays. This compound is reduced by viable cells in a colored soluble formazan salt.

The absorbance was measured on a Benchmark™ Microplate Reader (Biorad) (λ=490 nm) and compared with 12 cell control replicates (nor virus nor drug added) and 12 virus control wells (no drug added). Each assay was performed a minimum of three times. The 50% effective concentration (EC₅₀) was calculated from each dose response curve using the CurveExpert 1.3 software.

PBMCs isolated from the blood of healthy donor contain CD4⁺ T-lymphocytes which are one of the main targets of HIV in infected individuals.

Briefly, in the anti-HIV assay PBMCs, activated with phytohemagglutinin, were infected with HIV-1. 100 μl of infected PBMCs were seeded in 96-well plate at a concentration of 1 10⁵ cells/ml, and 100 μl of the different drugs dilutions were added to the cells. After 4 days of incubation at 37° C., 5% CO₂, cells cultures supernatants were collected. The amount of the viral capsid protein p24 in the supernatants was measured using the Innotest HIV Antigen mAb P24 kit (Innogenetics).

The results of tests are summarized in tables 4 to 7. From tables 4 and 5, it can be derived that the compounds according to the invention have good to excellent EC50 values and are able to inhibit HIV-1 activity in several tests performed on various cell lines such as P4, TZM-b1, MT-4 and PBMC. The best compounds (Z150, Z153) display a higher selectivity index (SI) than nevirapine and efavirenz due to their high antiviral activity combined with a low cytotoxicity. The demonstrated low cytotoxicity is a first indication that the compounds could be very useful in the treatment of HIV and especially HIV-1 infected individuals.

That some of the compounds, for instance Z150, are active on HIV-1 mutant strains resistant to Nevirapine (such as Cys188RT, Cys181RT, Asn103RT) is evident from table 6 and 7.

TABLE 4
Ex vivo anti-HIV activity (EC50), cytotoxicity (CC50) and
SI (selectivity index = CC50/EC50) for some compounds
according to the invention, the test being performed on
a P4 cell line with a WT (wild type) RT. Comparison with
Nevirapine and Efavirenz, two common RT inhibitors

| | EC₅₀ WT | CC₅₀ | |
|---|---|---|---|
| N^{o} | (μM) | (μM) | SI |
| M18 | 5.82 | >100 | >17 |
| Z12 | 3.09 | >100 | >32 |
| Z25 | 0.48 | 81 | 168 |
| Z30 | 0.52 | >100 | >192 |
| Z32 | 1.16 | 55 | 47 |
| Z33 | 1.03 | 54 | 52 |
| Z37 | 0.043 | 58 | 1349 |
| Z37A | 0.24 | 63 | 263 |
| Z37B | 0.085 | 72 | 847 |
| Z37inv | 0.036 | 64 | 1778 |
| Z45inv | 0.023 | 76 | 3304 |
| Z54 | 1.35 | 58 | 43 |
| Z57 | 1.82 | ND | ND |
| Z91inv | <0.001 | 37.2 | >37200 |
| Z114 | 0.007 | 8 | 1143 |
| Z121 | <0.001 | 27 | >27000 |
| Z122 | 0.001 | 27.5 | 27500 |
| Z150 | <0.001 | 66 | >66000 |
| Z153 | <0.001 | 54 | >54000 |
| Z154 | <0.001 | 80 | >80000 |
| Nevirapine | 0.029 | >100 | 3448 |
| Efavirenz | <0.001 | 40 | >40000 |

TABLE 5
Ex vivo anti-HIV activity (EC50) for some compounds according
to the invention, the test being performed on TZM-bl, MT4
and PBMC cell lines with a WT (wild type) RT. Comparison with
Nevirapine and Efavirenz, two common RT inhibitors

| | EC₅₀ | EC₅₀ | EC₅₀ |
|---|---|---|---|
| | TZM-bl | MT4 | PBMC |
| N^{o} | (μM) | (μM) | (μM) |
| Z37inv | 0.439 | ND | 0.054 |
| Z45inv | 0.197 | ND | 0.018 |
| Z91inv | <0.001 | <0.001 | <0.001 |
| Z114 | 0.011 | 0.136 | <0.001 |
| Z121 | ND | 0.014 | <0.001 |
| Z122 | ND | <0.001 | <0.001 |
| Z150 | <0.001 | <0.001 | ND |
| Z153 | <0.001 | ND | ND |
| Z154 | 0.003 | <0.001 | ND |
| Z167 | <0.001 | ND | ND |
| Nevirapine | 0.056 | 0.575 | 0.030 |
| Efavirenz | <0.001 | <0.001 | ND |

TABLE 6
Ex vivo anti-HIV activity (EC50) for some compounds according to the invention,
the test being performed on P4, TZM-bl or MT4 cell lines with mutant RT characterized
by a Cysteine for Tyrosine substitution at codon 188 (Cys188RT) or 181 (Cys181RT)
or by a Leucine for Isoleucine substitution at codon 100 (Ile100RT) in the RT.
Comparison with Nevirapine and Efavirenz, two common RT inhibitors.

| | Cys188RT | | | Cys181RT | | | Ile100RT | |
|---|---|---|---|---|---|---|---|---|
| | P4 | TZM-bl | MT4 | P4 | TZM-bl | MT4 | TZM-bl | MT4 |
| N° | (μM) | (μM) | (μM) | (μM) | (μM) | (μM) | (μM) | (μM) |
| Z37inv | 0.010 | ND | ND | 1.97 | 2.54 | ND | ND | ND |
| Z45inv | 0.008 | ND | ND | 0.496 | 3.2 | ND | 0.382 | ND |
| Z91inv | <0.001 | ND | 0.043 | 0.046 | 0.058 | 2.44 | 0.002 | 1.38 |
| Z114 | 0.003 | 0.001 | 1.68 | 0.087 | 0.411 | Inactive | 0.077 | Inactive |
| Z150 | <0.001 | <0.001 | 0.006 | 0.061 | 0.086 | 0.32 | 0.020 | 0.087 |
| Z153 | <0.001 | <0.001 | 0.033 | 0.108 | 0.083 | 0.78 | 0.029 | 0.187 |
| Z154 | <0.001 | ND | ND | ND | 0.288 | ND | 0.040 | ND |
| Z167 | ND | <0.001 | ND | ND | 0.007 | ND | ND | ND |
| Nevirapine | 1.635 | 2.72 | >5 | 10 | 8.3 | Inactive | 0.05 | 2.06 |
| Efavirenz | <0.001 | <0.001 | 0.001 | <0.001 | <0.001 | <0.001 | 0.017 | 0.178 |

TABLE 7
Ex vivo anti-HIV activity (EC50) for some compounds according to the invention,
the test being performed on P4, TZM-bl or MT4 cell lines with mutant RT characterized
by a Lysine for Asparagine substitution at codon 103 (Asn103RT), by a Valine
for Isoleucine substitution at codon 108 (Ile108RT) or by the double substitution
at codon 103 and 108 (Asn103/Ile108RT) in the RT. Comparison with Nevirapine
and Efavirenz, two common RT inhibitors.

| | Asn103RT | | | Ile108RT | | Asn103/Ile108RT | | |
|---|---|---|---|---|---|---|---|---|
| | P4 | TZM-bl | MT4 | TZM-bl | MT4 | F4 | TZM-bl | MT4 |
| N° | (μM) | (μM) | (μM) | (μM) | (μM) | (μM) | (μM) | (μM) |
| Z37inv | ND | ND | ND | 1.09 | ND | ND | ND | ND |
| Z45inv | ND | ND | ND | ND | ND | 19.2 | ND | ND |
| Z91inv | 0.089 | 0.355 | 0.98 | <0.001 | 0.072 | 0.18 | ND | 9.9 |
| Z114 | 0.643 | 0.584 | inactive | 0.012 | 12 | 1.93 | ND | inactive |
| Z150 | ND | 0.011 | <0.001 | 0.004 | <0.001 | 0.027 | 0.158 | 0.958 |
| Z153 | ND | 0.042 | 0.15 | 0.001 | 0.008 | 0.070 | 0.366 | 3.48 |
| Z154 | ND | 0.029 | ND | 0.014 | ND | 0.088 | 0.231 | ND |
| Z167 | ND | ND | ND | ND | ND | ND | 0.129 | ND |
| Nevirapine | 1.71 | 2.1 | >25 | 0.091 | 1.27 | 6.25 | ND | inactive |
| Efavirenz | ND | 0.035 | 0.005 | <0.006 | <0.001 | 0.009 | 0.150 | 1.19 |

The compounds according to the present invention could be administrated orally to humans in a dosage range of 1 to 100 mg/kg body weight in divided doses. It will be understood, however, that the specific dose level and frequency of dosage for any particular patient may vary and will depend upon a variety of factors including the activity of the compound employed, its metabolic stability and length of action, as well as the age, the weight and the general health of the patient at the time of the administration, the rate of excretion, the other drugs used, and the host undergoing therapy. It falls within the skills of an artisan to determine the concentration of drugs that should be used in HIV-1 treatment.

The compounds of the present invention can be used for the preparation of medicaments such as therapeutic compositions for the treatment of HIV-1 related diseases. The compounds can be used alone (in pure form, as salt or as prodrug), or as mixtures of several compounds, whether or not in combination with other compounds active against HIV-1 infections.

Such anti-viral agents include other NNRTIs such as Nevirapne, Efavirenz, Delavirdine, Capravirine and the like as well as NRTIs, protease inhibitors, fusion/binding inhibitors, integrase inhibitors, pyrophosphate analogue RT inhibitors and/or HIV vaccines. The above list is not exhaustive and may include any other anti-viral, anti-infective, antibiotic as well as any immunomodulator. The effect can be additive and/or synergistic.

The compounds according to the invention and mixtures thereof with any other therapeutic and/or pharmaceutical agent can be used in pharmaceutical compositions comprising an acceptable diluent and/or carrier. These are known to a skilled person.

Administration in the case of combinations can be together or consecutively whereby the interval can range from minutes to hours. It is evident that other applications than oral applications are possible, for instance in the case of combination with a therapeutic and/or prophylactic vaccine. It is further evident that the compounds according to the inventions can be applied under any form that does not preclude their activity, such forms including pills, liquids, powders, pastes and any other form or formulation known in the art.

### Example 11

### Comparison of Some Compounds According to the Invention with Compounds Disclosed in Prior Art and Influence of the R2 Type on the Activity of the Compound

Compounds according to the present invention were compared with compounds known in the art. Provided that any group that is linked to position 4 of the pyridinone ring is hereby referenced as “R2”, and X is defined as the “spacer” between the two groups, the following can be concluded

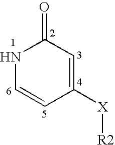

Compounds disclosed in patents EP 0 462 800 and EP 0 481 802 differ by their substitution in position 3 and differ by their substitution in position 4 (no spacer between the pyridinone and R2 in the compounds disclosed in EP 0462 800 and EP 0 481 802 whereas all compounds of the present invention have such a spacer).

The compounds disclosed in patent EP 0 462 808 differ by their substitution in position 3 (all possess a phtaloyl group on this position). They are further not substituted in position 4.

The compounds disclosed in WO97/05113 and publication of Dolle differ by their substitution in position 4. Only arylthio and arylamino groups are considered, whereas compounds of the invention do not feature such groups in position 4.

The compounds disclosed in WO99/55676 differ by their substitution in position 3. Only amino or alkylamino groups have been considered. None of these chemical functions is present in the invention.

Compounds disclosed in International patent application WO02/24650 are of the above the compounds most closely related with those of the present invention, the compounds having a spacer between the pyridinone ring and R2, the R2 groups may be comparable at first sight. However, the specific compounds disclosed in WO02/24650 to have anti-HIV activity all feature an aryl substituent at position 4 of the pyridinone ring, unlike compounds of the present invention. No compound bearing a C₇₊ cycloalkyl or a substituted cycloalkyl in position 4 is disclosed and/or claimed in International patent application WO02/24650.

The following table 8 demonstrates the interest of for instance C₇₊ cycloalkyls or substituted cycloalkyls as evident from the EC50 value:

TABLE 8
Effect of the R2 substituent on EC₅₀ values

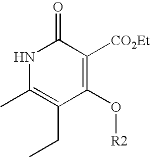

| | EC₅₀ | EC₅₀ |
|---|---|---|
| R2 | WT (μM) | Cys188RT (μM) |
| 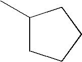 | 1.91 | ND |
| 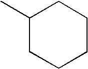 | 0.77 | 6.5 |
| 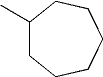 | 0.48 | 3.28 |
| (formula VII, Z25) | | |
| 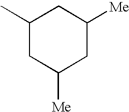 | 0.043 | 0.006 |
| (formula II, Z37) | | |

ND, not determined

As shown in Table 8, replacement of a cyclopentyl or cyclohexyl by a cycloheptyl leads to an increase in antiviral potency of the compounds.

Replacement of a cyclohexyl by a m,m-dimethyl-cyclohexyl leads to a 16-fold increase in antiviral potency.

Furthermore, compounds Z25 and Z37 are more active on Cys188 mutant strains than the cyclohexyl derivative.

It goes beyond any doubt that the above examples are sufficient to demonstrate that the problem of providing alternative compounds active against HIV-1, with pronounced NNRTI activity, is solved by the compounds according to the invention which are novel and inventive.

Advantageously, compounds according to the invention can be active against HIV-1 strains that are resistant to NNRTIs currently used such as Nevirapine.

1. A 5-ethyl-6-methyl-2-pyridinone deriva-tive compound according to general formula I, 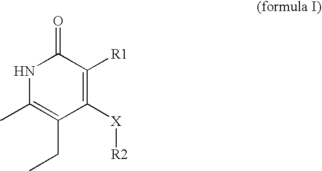
wherein
X O, S, NH, C═O, (CₙH₂ₙ), (CₙH₂ₙ)O, O(CₙH₂ₙ), (CₙH₂ₙ)S, S(CₙH₂ₙ)with n=1-4 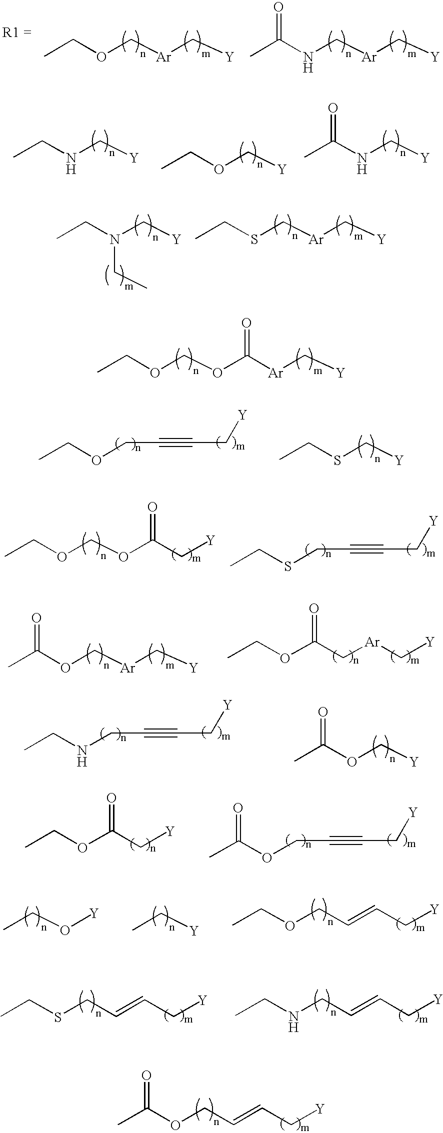
with n, m=0-8
Ar=Aromatic ring selected from: phenyl, pyridyl, thiazolyl, furanyl, thiophenyl, benzofuranyl, benzothiophenyl, benzothiazolyl, imidazolyl, indolyl,
each optionally substituted with up to 4 substituants selected from:
halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido
Y=alkyl, amino, nitro or
Y═H, halo, alkylamino, dialkylamino, nitrile, hydroxy, C₁₋₆alkyloxycarbonyl, C₁₋₆alkylcarbonyloxy, C₅₋₇ cycloalkyl optionally substituted with up to 4 substituants selected from:
halo, hydroxy, C₁₋₄ alkyl, C₁₋₄ alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, nitrile;
or Y can be: 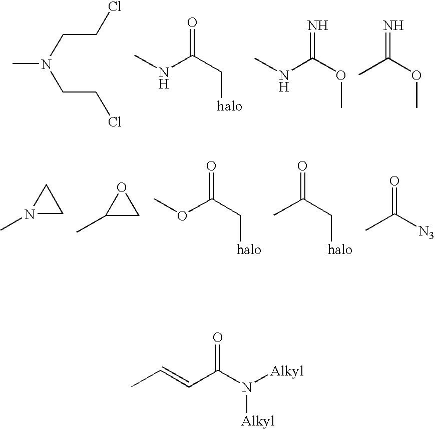
R2=C₇₋₉ cycloalkyl;
C₅₋₈ cycloalkyl substituted with up to 4 substituants;
C₅₋₈cycloalkenyl optionally substituted with up to 4 substituants;
C₅₋₈aliphatic heterocycle optionally substituted with up to 4 substituants;
C₆₋₉bridged cycloalkyl optionally substituted with up to 4 substituants;
C₆₋₉bridged cycloalkenyl optionally substituted with up to 4 substituants;
substituants selected from:
halo, hydroxy, C₁₋₄ alkyl, C₁₋₄alkoxy, C₁₋₄ hydroxyalkyl, C₁₋₄ alkylamino, amino, C₁₋₄ aminoalkyl, C₁₋₄ alkylcarbonyl, C₁₋₄ dialkylamino, azido, CN;

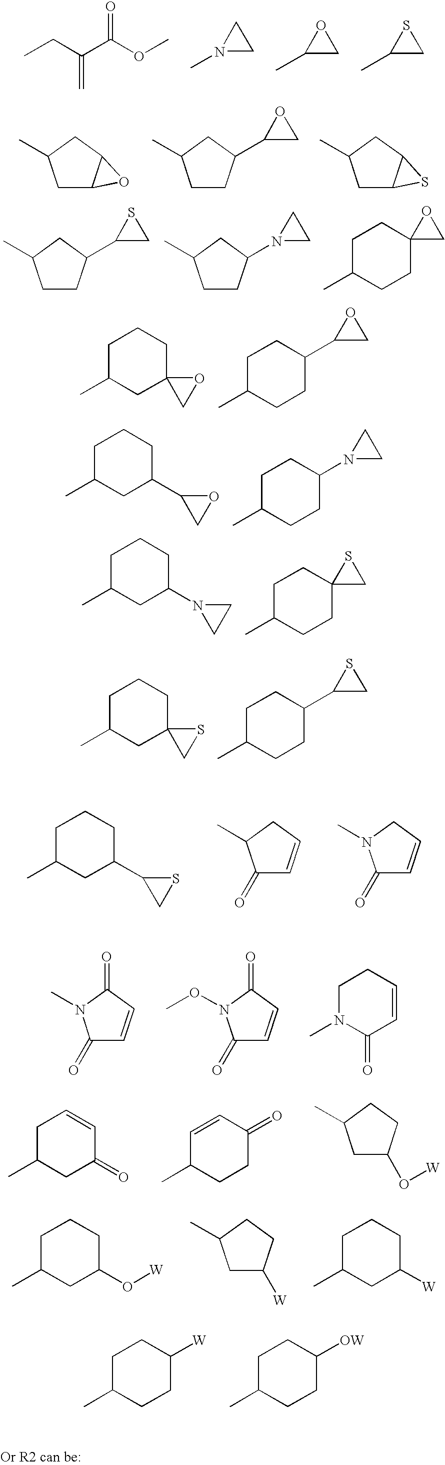

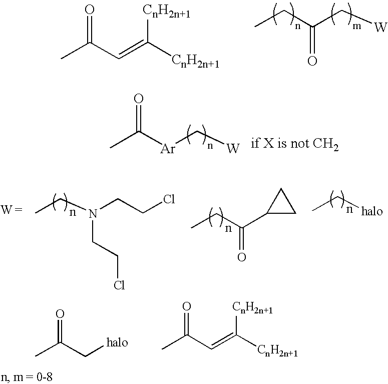

2. The compound according to claim 1 which comprises a substituted cycloalkyl group as R2 in position 4 of the pyridinone ring.

3. The compound according to claim 2 wherein the substituted cycloalkyl group is a 3,5-dimethylcyclohexyl

4. The compound according to claim 1 which comprises a C7-9 cycloalkyl group as R2 in position 4 of the pyridinone ring.

5. The compound according to claim 1 wherein R2 has the formula XII 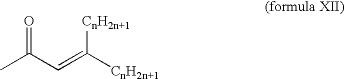
with n=0-8.

6. The compound according to claim 1 which is selected from the group consisting of M18, Z12, Z25, Z30, Z32, Z33, Z37, Z37inv, Z53, Z54, Z55, Z57, Z45inv, Z91inv, Z96inv, Z114, Z121, Z122, Z150, Z153, Z154 and Z167, wherein X, R1 and R2 are as indicated below:

| N° | X | R1 | R2 |
|---|---|---|---|
| M18 | O | CO₂Et | 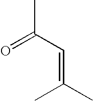 |
| Z12 | O | CO₂Et | 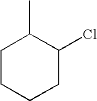 |
| Z25 | O | CO₂Et | 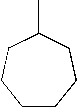 |
| Z30 | O | CO₂Et | 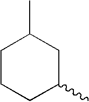 |
| Z32 | O | CH₂OH | 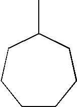 |
| Z33 | O | 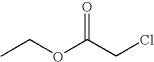 | 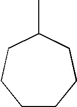 |
| Z37 | O | CO₂Et | 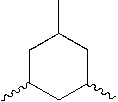 |
| Z53 | O | 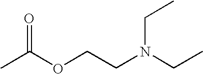 | 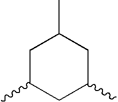 |
| Z54 | O | CO₂Et | 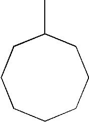 |
| Z55 | O | CO₂Et | 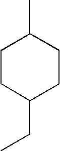 |
| Z57 | 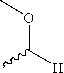 | CO₂Et | 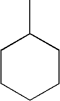 |
| Z45 inv | O | CH₂OH | 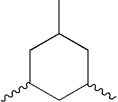 |
| Z91 inv | O | NO₂ | 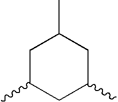 |
| Z96 inv | O | NH₂ | 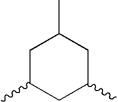 |
| Z114 | O | CH₂SCOMe | 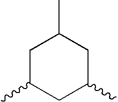 |
| Z121 | O | CH₂S(CH₂)₂OH | 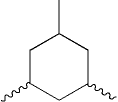 |
| Z122 | O | CH₂S(CH₂)₂OCOCH₂Cl | 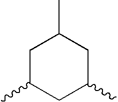 |
| Z150 | O | NMe₂ | 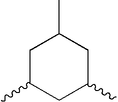 |
| Z153 | O | CH₂N₃ | 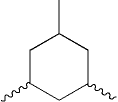 |
| Z154 | O | Me | 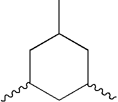 |
| Z167 | O | Et | 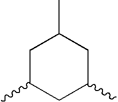 |

7. A compound according to claim 1, with X═O, R1=CO₂Et and

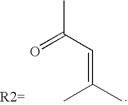

8. A pharmaceutical composition comprising the compound according to the claim 1 and an acceptable carrier and/or diluent.

9. The composition according to claim 8 further comprising another anti-viral agent.

10. The composition according to claim 9, wherein the said anti-viral agent is Nevirapine.

11. A method for the treatment and/or the prevention of HIV-1 infections in a mammal, which comprises the step of administrating the compound of claim 1 or the composition of claim 8 to the mammal.

12. The method according to the claim 11 for the treatment and/or prevention of HIV-1 infections by a strain resistant to at least one anti-viral agent.

13. The method of claim 12 wherein said anti-viral agent is Nevirapine.

14. A method for obtaining an irreversible anti-HIV-1 compound, which comprises the steps of:
selecting an anti-HIV-1 compound that interacts with a binding site of an HIV-1 enzyme,
introducing a chemical modification in the structure of the anti-HIV-1 compound that allows the formation of at least one covalent bond between the compound and an amino acid of said HIV-1 enzyme.

15. The method of claim 14, wherein the HIV I binding site is the allosteric site of HIV I reverse transcriptase.

16. The method of claim 14 wherein the anti-HIV-1 compound is an NNRTI.

17. An irreversible NNRTI obtainable by the method of claim 16.

18. The irreversible NNRTI according to claim 17 which is a compound (Z122) according to formula I with X═O, R1=CH₂S(CH₂)₂OCOCH₂Cl and [chemical structure]
